(19) United States
(12) Patent Application Publication
(10) Pub. No.: US 2016/0174815 A1
(43) Pub. Date: Jun. 23, 2016

(54) INTRODUCTION DEVICE

(71) Applicant: OLYMPUS CORPORATION, Tokyo (JP)

(72) Inventor: Yasuhiro OKAMOTO, Hachioji-shi (JP)

(73) Assignee: OLYMPUS CORPORATION, Tokyo (JP)

(21) Appl. No.: 15/055,892

(22) Filed: Feb. 29, 2016

Related U.S. Application Data
(63) Continuation of application No. PCT/JP2014/075912, filed on Sep. 29, 2014.

(30) Foreign Application Priority Data
Nov. 20, 2013 (JP) 2013-240319

(51) Int. Cl. A61B 1/005

(52) U.S. Cl. CPC A61B 1/0052

(57) ABSTRACT

An introduction device includes an operation section including a first surface and a second surface; a bending section capable of bending in a first direction and a second direction crossing the first direction; a first rotary body configured to be operated at a time of bending the bending section in the first direction; and a second rotary body configured to be rotatable about a second shaft portion extending in a direction along the first shaft portion at a position which is closer to the thumb than the first shaft portion, to be provided on the second surface side within an operation range of the thumb, and to be operated at a time of bending the bending section in the second direction.

### CROSS-REFERENCE TO RELATED APPLICATIONS

This application is a Continuation Application of PCT Application No. PCT/JP2014/075912, filed Sep. 29, 2014 and based upon and claiming the benefit of priority from prior Japanese Patent Application No. 2013-240319, filed Nov. 20, 2013, the entire contents of all of which are incorporated herein by reference.

### BACKGROUND OF THE INVENTION

1. Field of the Invention

The present invention relates to an introduction device which is inserted into a cavity.

2. Description of the Related Art

In general, an introduction device for introduction into a cavity, such as an endoscope, includes, for example, an insertion section with flexibility which is inserted into a subject in order to observe, treat, etc. a diseased part in the subject; and an operation section for performing an operation for bending the insertion section in a UD direction and an RL direction. The operation section includes a UD angle knob for bending the insertion section in the UD direction, and an RL angle knob for bending the insertion section in the RL direction.

In this introduction device, the UD angle knob and RL angle knob are operated, and the insertion section is bent in the UD direction and RL direction, and thereby a in the UD direction and RL direction, and thereby a diseased part can be observed, treated, etc. In a general introduction device, in many cases, the bending operations in the UD direction and RL direction are performed by the force of a finger via wires or the like which are passed thought the insertion section. In such cases, the operation input direction of the UD angle knob and RL angle knob is, in most cases, disposed along the longitudinal direction of a grip section. In addition, many therapy techniques and observation methods have been established thus far for introduction devices having such general disposition of the UD angle knob and RL angle knob, and these are compiled in manuals.

In addition, there is an endoscope in which the bending of a bending section in the UD direction and RL direction is driven by a motor.

For example, in an endoscope of PCT International Publication No. 2012-074013, bending of the insertion section in the up-and-down (UD) direction is performed by a manual operation, and bending in the right-and-left (RL) direction is automatically operated by driving of a motor. The operation section includes a knob for an operation in the up-and-down (UD) direction, and a dial for an operation in the right-and-left (RL) direction.

### CITATION LIST

### Patent Literature

Patent document 1: PCT International Publication No. 2012-074013
Patent document 2: Jpn. Pat. Appln. KOKAI Publication No. 2006-320501

### BRIEF SUMMARY OF THE INVENTION

In order to achieve the object, an introduction device according to one embodiment of the invention includes an operation section including a first surface, and a second surface which neighbors the first surface and is located on a side of a thumb of a holding hand; a bending section capable of bending in a first direction and a second direction crossing the first direction; a first rotary body configured to be rotatable about a first shaft portion projecting from the first surface in a direction crossing a longitudinal axis of the operation section, and to be operated at a time of bending the bending section in the first direction; and a second rotary body configured to be rotatable about a second shaft portion extending in a direction along the first shaft portion at a position which is closer to the thumb than the first shaft portion, to be provided on the second surface side within an operation range of the thumb, and to be operated at a time of bending the bending section in the second direction.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view illustrating the entire structure of an endoscope device of a first embodiment.

FIG. 2 is a front view illustrating, from an end face side, a distal rigid section of the endoscope device shown in FIG. 1.

FIG. 3 is a cross-sectional view illustrating bend pieces, first wires and second wires, which are disposed within a bending section of the endoscope device shown in FIG. 1.

FIG. 4 is a cross-sectional view taken along line F4-F4 in FIG. 3.

FIG. 5 is a front view illustrating, from a first surface side (front-surface side), an operation section of the endoscope device shown in FIG. 1.

FIG. 6 is a side view illustrating, from a second surface side (side-surface side), the operation section of the endoscope device shown in FIG. 5.

FIG. 7 is a cross-sectional view, taken along line F7-F7 in FIG. 5, illustrating the endoscope device shown in FIG. 5.

FIG. 8 is a front view illustrating, from the first surface side (front-surface side), an operation section of an endoscope device of a second embodiment.

FIG. 9 is a side view illustrating, from the second surface side (side-surface side), the operation section of the endoscope device shown in FIG. 8.

FIG. 10 is a front view illustrating, from the first surface side (front-surface side), an operation section of an endoscope device of a third embodiment.

FIG. 11 is a side view illustrating, from the second surface side (side-surface side), the operation section of the endoscope device shown in FIG. 10.

FIG. 12 is a front view illustrating, from the first surface side (front-surface side), an operation section of an endoscope device of a fourth embodiment.

FIG. 13 is a side view illustrating, from the second surface side (side-surface side), the operation section of the endoscope device shown in FIG. 12.

FIG. 14 is a cross-sectional view taken along line F14-F14 in FIG. 12, illustrating the endoscope device shown in FIG. 12.

FIG. 15 is a front view illustrating, from the first surface side (front-surface side), an operation section of an endoscope device of a fifth embodiment.

FIG. 16 is a side view illustrating, from the second surface side (side-surface side), the operation section of the endoscope device shown in FIG. 15.

FIG. 17 is a cross-sectional view taken along line F17-F17 in FIG. 15, illustrating the endoscope device shown in FIG. 15.

FIG. 18 is a front view illustrating, from the first surface side (front-surface side), an operation section of an endoscope device of a sixth embodiment.

FIG. 19 is a schematic view illustrating a state in which at least a first part of a bending section of an endoscope shown in FIG. 18 is bent in a U direction or a D direction, and a state in which the first part and a second part of the bending section are bent in the U direction or D direction.

FIG. 20 is a side view illustrating, from the second surface side (side-surface side), the operation section of the endoscope device shown in FIG. 18.

FIG. 21 is a front view illustrating, from the first surface side (front-surface side), an operation section of an endoscope device of a seventh embodiment.

FIG. 22 is a side view illustrating, from the second surface side (side-surface side), the operation section of the endoscope device shown in FIG. 21.

FIG. 23 is a front view illustrating, from the first surface side (front-surface side), an operation section of an endoscope device of an eighth embodiment.

FIG. 24 is a side view illustrating, from the second surface side (side-surface side), the operation section of the endoscope device shown in FIG. 23.

FIG. 25 is a front view illustrating, from the first surface side (front-surface side), an operation section of an endoscope device of a ninth embodiment.

FIG. 26 is a side view illustrating, from the second surface side (side-surface side), the operation section of the endoscope device shown in FIG. 25.

FIG. 27 is a cross-sectional view taken along line F27-F27 in FIG. 25, illustrating the endoscope device shown in FIG. 25.

### DESCRIPTION OF EMBODIMENTS

### First Embodiment

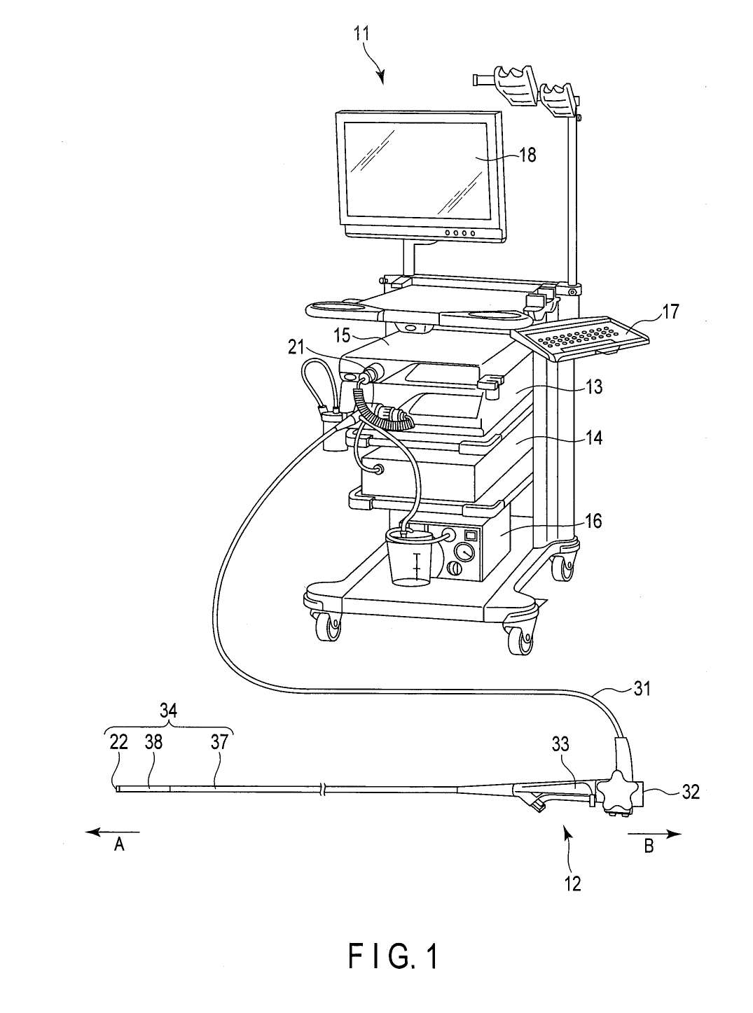

FIG. 1 illustrates the entire structure of an endoscope device of the present invention. As illustrated in FIG. 1, an endoscope device 11 includes an endoscope 12, a control device 13, a light source device 14, an image capturing device 15, an air-feed/water-feed/suction device 16, a keyboard 17, a monitor 18, and an actuator unit 21.

The light source device 14 supplies, under the control of the control device 13, light to illumination lenses 23 disposed at a distal rigid section 22 (to be described later) of the endoscope 12. The air-feed/water-feed/suction device 16 feeds, under the control of the control device 13, air/water to a nozzle 24 disposed at the distal rigid section 22 of the endoscope 12, and sucks a liquid or tissue from a living body via the nozzle 24. The image capturing device 15 processes, under the control of the control device 13, an image of a subject, which was captured through an objective lens 25 at the distal rigid section 22 of the endoscope 12, and displays the processed image on the monitor 18.

The control device 13 is connected to a rotation detection sensor 26 (see FIG. 7) which is built in an operation section 32 (to be described later) of the endoscope 12. The rotation detection sensor 26 detects a rotational direction and a rotation amount of a second rotary body 52, and transmits a detection signal to the control device 13. The control device 13 operates the actuator unit 21 in accordance with the rotation amount detected by the rotation detection sensor 26, and bends the bending section 38 in an R direction or L direction.

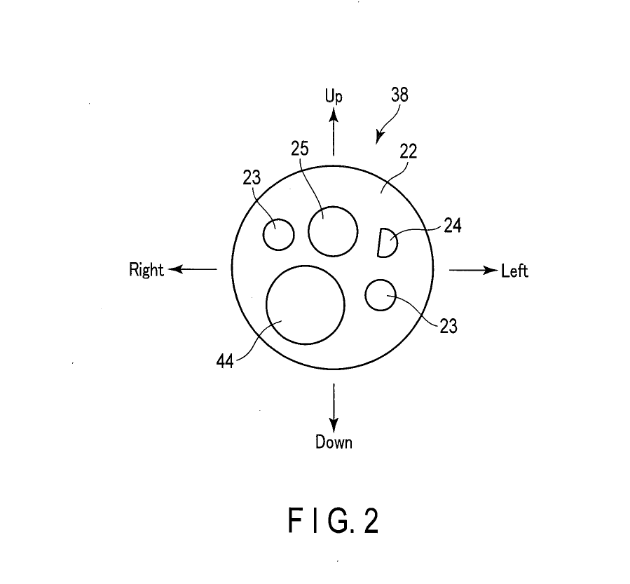

The actuator unit 21 can apply a driving force so as to bend the bending section 38 (to be described later) of the endoscope 12 in the R direction (“Right” side indicated in FIG. 2) and L direction (“Left” side indicated in FIG. 2). The actuator unit 21 is composed of, for example, a motor such as a servo motor. The actuator unit 21 is built in a connector of a universal cord 31 (a connector connected to the control device 13).

As illustrated in FIG. 1, the endoscope 12 includes the universal cord 31, operation section 32, a grip section 33 which neighbors the operation section 32 and provided integral with the operation section 32, and an insertion section 34 which extends from the grip section 33 and is inserted in a cavity (subject). The endoscope 12 is an example of the introduction device.

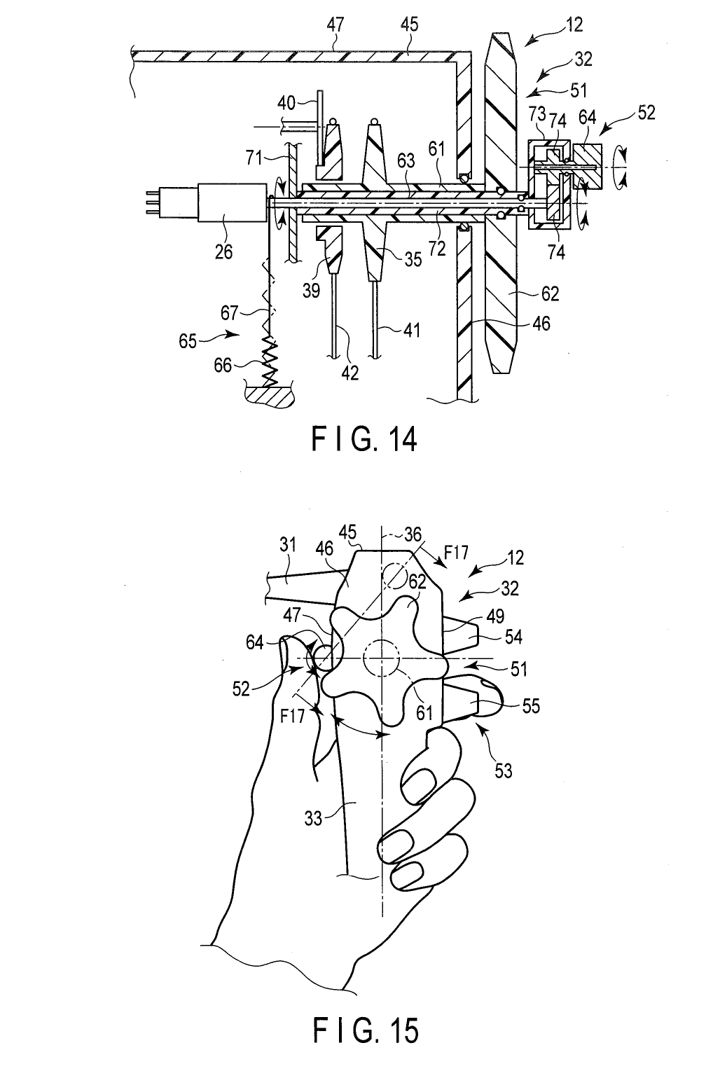

The endoscope 12 is connected to the control device 13, light source device 14, image capturing device 15 and air-feed/water-feed/suction device 16 via the universal cord 31. A flexible shaft (not shown) is passed through the universal cord 31. The driving force of the actuator unit 21 is transmitted via the flexible shaft (torque shaft) and a gear 40 and a second sprocket 39 (see FIG. 14), which are provided within the operation section 32 so as to receive force transmitted from this shaft, to a pair of second wires 42 which are wound around the second sprocket 39.

The insertion section 34 is provided along a longitudinal axis 36 (see FIG. 1, FIG. 5, etc.) which passes through substantially the center of the operation section 32. In FIG. 1, arrow A indicates a distal-end direction of the longitudinal axis 36, and arrow B indicates a proximal-end direction of the longitudinal axis 36. The insertion section 34 includes a flexible section 37 which is long and narrow and has flexibility, a bending section 38 provided at a distal end of this flexible section 37, and a distal rigid section 22 provided at a distal end of this bending section 38.

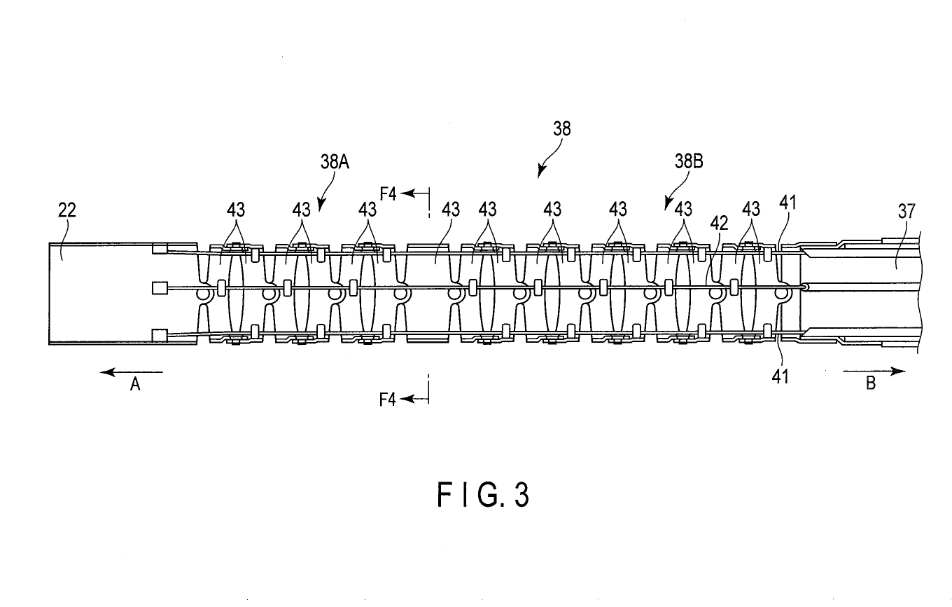

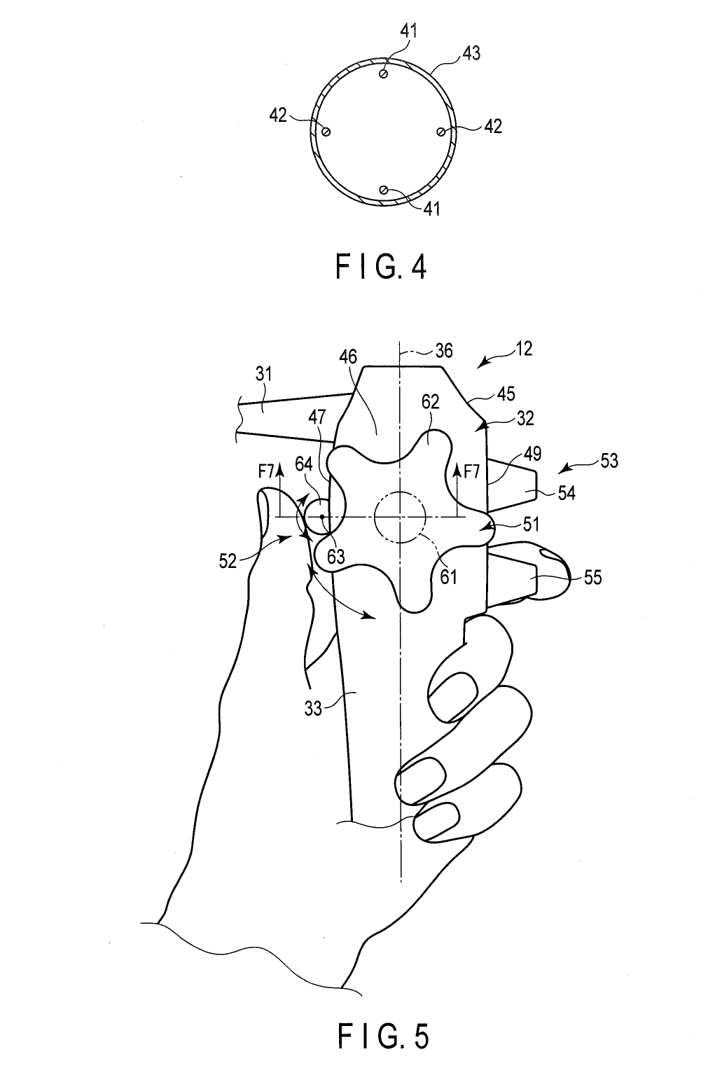

As illustrated in FIG. 3 and FIG. 4, a pair of first wires 41 for bending the bending section 38 in the U direction (“Up” side indicated in FIG. 2) and D direction (“Down” side indicated in FIG. 2) and a pair of second wires 42 for bending the bending section 38 in the R direction and L direction are passed through the inside of the flexible section 37 and bending section 38. The bending section 38 includes a plurality of bend pieces 43 which are arranged along the longitudinal axis 36 of the insertion section 34.

As illustrated in FIG. 2, the distal rigid section 22 is provided with an objective lens 25, a treatment instrument insertion channel 44, illumination lenses 23, and a nozzle 24 which is capable of supplying water and air for cleaning the distal end face of the distal rigid section 22, and sucking a liquid or tissue in the living body.

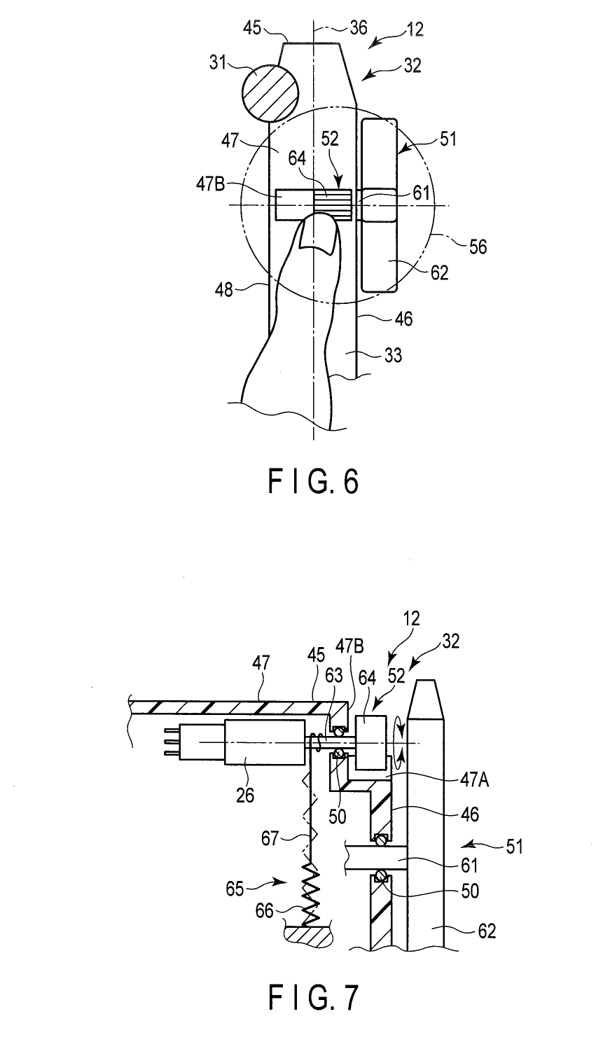

As illustrated in FIG. 5 and FIG. 6, the operation section 32 includes a case 45 which is formed of, e.g. a synthetic resin material so as to have an inside space; a first rotary body 51 which is provided on a first surface 46 side of the case 45; a second rotary body 52 which is provided on a second surface 47 side of the case 45; a button section 53 which is provided on a fourth surface 49 side of the case 45; and a rotation detection sensor 26 (see FIG. 7) which is provided within the case 45.

As illustrated in FIG. 5 and FIG. 6, the case 45 includes a first surface 46, a second surface 47 which is provided at a position neighboring the first surface 46, a third surface 48 which is opposed to the first surface 46, and a fourth surface 49 which is opposed to the second surface 47. The first surface 46 extends in a direction crossing a first shaft portion 61 of the first rotary body 51 which will be described later. The second surface 47 is located on the thumb side of the hand (left hand) that holds the operation section 32, and extends in a direction crossing (perpendicular to) the first surface 46 from an outer edge portion of the first surface 46. It can be said, in other words, that the second surface 47 extends from the outer edge portion of the first surface 46 along a direction in which the first shaft portion 61 of the first rotary body 51 (to be described later) extends. As illustrated in FIG. 7, the second surface 47 is provided with a recess portion 47A in which a part of a second dial portion 64 of the second rotary body 52 is to be disposed.

Each of the first surface 46 and second surface 47 may be a flat surface or a curved surface. When the first surface 46 and second surface 47 are curved surfaces, it is preferable that these surfaces are formed in a manner to bulge to the outside relative to the longitudinal axis 36. As illustrated in FIG. 7, O-rings 50 are interposed between the first shaft portion 61 and case 45 and between the second shaft portion 63 and case 45, thereby keeping water-tight the inside of the case.

The rotation detection sensor 26 is composed of, for example, a potentiometer. However, another kind of sensor (e.g. a rotary encoder) may be used if the sensor can detect the rotation amount of the second shaft portion 63. The rotation detection sensor 26 reads the rotational angle of the second dial portion 64 via the second shaft portion 63 of the second rotary body 52, and detects the rotational direction and rotation amount of the second dial portion 64.

As illustrated in FIG. 5, the button section 53 includes a first button 54 (air-feed/water-feed button) for feeding air and water to the distal rigid section 22 of the endoscope 12 via the nozzle 24, and a second button 55 (suction button) for sucking at the distal rigid section 22 of the endoscope 12 via the nozzle 24.

The first rotary body 51 is a so-called UD knob which is operated to bend the bending section 38 at a time of bending the bending section 38 in a U direction and a D direction, that is, at a time of bending the bending section 38 in two directions. If a doctor rotates the first rotary body 51 about the first shaft portion 61 by a finger or the like, the bending section 38 is bent in accordance with the rotation amount in the U direction or D direction (these directions are comprehensively referred to as “first direction”) illustrated in FIG. 2.

The first rotary body 51 includes a first shaft portion 61 which projects from the first surface 46 of the case 45 of the operation section 32 and is provided rotatable relative to the case 45; a first dial portion 62 which is fixed to a distal end portion of the first shaft portion 61 and is rotatable about the first shaft portion 61; and a first sprocket 35 (see FIG. 14) which is provided within the case 45 and is fixed to a proximal end portion of the first shaft portion 61. The first shaft portion 61 extends in a direction crossing the longitudinal axis 36 of the operation section 32. The first dial portion 62 has a substantially star-like shape, and includes, for example, five claws. The first wires 41 for bending the bending section 38 in the U direction and D direction are wound around the first sprocket 35. The first sprocket 35 and first wires 41 constitute a first bending mechanism for manually bending the bending section 38 in the first direction.

In the endoscope device 11 of the present embodiment, there is no mechanism for bending the bending section in the U direction and D direction by electric driving of a motor or the like. However, like the bending in the R direction and L direction to be described later, the bending section 38 may be bent by electric driving in the U direction and D direction by providing the actuator unit 21 such as a motor. In addition, it is preferable that the first shaft portion 61 of the first rotary body 51 is formed to cross the longitudinal axis 36 (for example, to be perpendicular to the longitudinal axis 36).

The second rotary body 52 is a so-called RL dial which is operated to bend the bending section 38 at a time of bending the bending section 38 in an R direction and an L direction, that is, at a time of bending the bending section 38 in two directions. If the doctor rotates the second rotary body 52 about the second shaft portion 63, the actuator unit 21 is driven, and the bending section 38 is bent by the driving force of the actuator unit 21. In accordance with the rotation amount of the second rotary body 52, the bending section 38 is bent in the R direction and L direction (these directions are comprehensively referred to as “second direction”) illustrated in FIG. 2. In the present embodiment, the second direction crosses (interests at right angles with) the first direction.

As illustrated in FIG. 5 and FIG. 6, the second rotary body 52 is provided near the first rotary body 51 on the second surface 47 side, and is provided within an operation range 56 of the thumb of the left hand that holds the operation section 32. In this embodiment, the second rotary body 52 is provided at a position between the first surface 46 and third surface 48. It can be said, in other words, that the second rotary body 52 is provided within the range of the thickness dimension of the case 45 in the direction of extension of the first shaft portion 61.

As illustrated in FIG. 5 and FIG. 7, the second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second rotary body 52 is provided on the second surface 47 side.

As illustrated in FIG. 5, the second shaft portion 63 is provided at substantially the same position as the first shaft portion 61 in the longitudinal axis 36 direction. As illustrated in FIG. 5 and FIG. 7, the second shaft portion 63 is provided at a position which is closer to the thumb of the hand (left hand) that holds the operation section 32, than the first shaft portion 61. The second shaft portion 63 extends in a direction along the direction of extension of the first shaft portion 61 and, to be more specific, extends substantially parallel to the first shaft portion 61.

The second dial portion 64 has a columnar shape. The second dial portion 64 has a smaller diameter than the first dial portion 62. The peripheral surface of the second dial portion 64 is provided with, for example, knurling-like recess and projections. A part of the second dial portion 64 is disposed within the recess portion 47A provided in the second surface 47, and the other part of the second dial portion 64 is exposed to the outside. A proximal end portion of the second shaft portion 63 is connected to the rotation detection sensor 26 in the inside of the case 45.

The actuator unit 21, flexible shaft, gear 40, second sprocket 39 and second wires 42 constitute a second bending mechanism for bending the bending section 38 in the second direction.

As illustrated in FIG. 7, the return-to-neutral mechanism 65 includes a tension coil spring 66, and a tension wire 67 which is interposed between the second shaft portion 63 and tension coil spring 66. By the action of the tension coil spring 66, the return-to-neutral mechanism 65 can apply a proper return-to-neutral force to the second shaft portion 63 and second dial portion 64.

In the meantime, in the present embodiment, although the description is given of the example in which the bending section 38 is bent by electric driving with respect to the R direction and L direction, it is possible to adopt such a configuration that the bending section 38 is manually bent in the R direction and L direction, like the bending mechanism for bending the bending section 38 in the U direction and D direction.

Next, the operation of the endoscope device 11 of the present embodiment is described. A doctor holds the operation section 32 by, for example, the left hand. As illustrated in FIG. 5, etc., the universal cord 31 is placed at a position between the left thumb and index finger, the inside of the thumb is placed on the claw of the first dial portion 62, and the grip section 33 is supported by the middle finger, ring finger and little finger. In addition, the inside of the left index finger is disposed at such a position as to able to operate the first button 54 (air-feed/water-feed button) and second button 55 (suction button). Besides, by holding the insertion section 34 by the right hand and inserting the insertion section 34 into a cavity, a desired examination or treatment is performed.

When the doctor wishes to bend the bending section 38 in one of the U direction and D direction (first direction) illustrated in FIG. 2, the doctor rotates the first dial portion 62 clockwise or counterclockwise by, for example, the inside of the thumb of the left hand. Thereby, the first sprocket 35, which is fixed to the first shaft portion 61 in the inside of the operation section 32, rotates, and one of the paired first wires 41, which are wound around the first sprocket 35, is pulled toward the proximal end side of the operation section 32. Thus, the bending section 38 bends in either the U direction or D direction. Specifically, if the first rotary body 51 is rotated clockwise in FIG. 5, the bending section 38 bends in the D (downward) direction. If the first rotary body 51 is rotated counterclockwise, the bending section 38 bends in the U (upward) direction.

On the other hand, when the doctor wishes to bend the bending section 38 in one of the R direction and L direction (second direction) illustrated in FIG. 2, the doctor rotates, as illustrated in FIG. 5, etc., the second dial portion 64 clockwise or counterclockwise by the inside of the thumb. As illustrated in FIG. 5, for example, if the second dial portion 64 is rotated clockwise, the bending section 38 bends in the R (right) direction. If the second dial portion 64 is rotated counterclockwise, the bending section 38 bends in the L (left) direction. In the present embodiment, since the second rotary body 52 is located near the first rotary body 51, the thumb of the doctor easily reaches the second rotary body 52, and the operability becomes good.

The rotation detection sensor 26 transmits an electric signal, which corresponds to the rotation amount of the second dial portion 64, to the control device 13. The control device 13 operates the actuator unit 21, and the actuator unit 21 transmits a torque (rotational force) to the paired second wires 42 via the flexible shaft, gear 40 and second sprocket 39. One of the second wires 42 is pulled toward the proximal end side of the operation section 32, and the bending section 38 bends in either the R direction or L direction. In addition, if the doctor releases the thumb from the second rotary body 52, the second rotary body 52 is returned to the neutral position by the return-to-neutral mechanism 65. In accordance with this, the actuator unit 21 operates, and the bending section 38 also returns to the neutral position from the bent state in the R direction or L direction.

According to the first embodiment, the introduction device includes the operation section 32 including the first surface 46, and the second surface 47 which neighbors the first surface 46 and is located on the thumb side of the holding hand; the bending section 38 capable of bending in the first direction and second direction; the first rotary body 51 configured to be rotatable about the first shaft portion 61 projecting from the first surface 46 in a direction crossing the longitudinal axis 36 of the operation section 32, and to be operated at a time of bending the bending section 38 in the first direction; and the second rotary body 52 configured to be rotatable about the second shaft portion 63 extending in a direction along the first shaft portion 61 at a position which is closer to the thumb than the first shaft portion 61, to be provided on the second surface 47 side within the operation range 56 of the thumb, and to be operated at a time of bending the bending section 38 in the second direction.

According to this structure, since the second shaft portion 63 is provided at a position closer to the thumb than the first shaft portion 61, the second rotary body 52 can be disposed at a position closer to the thumb than the first rotary body 51. Thereby, even in the case of a doctor with a small hand, the hand easily reaches the second rotary body 52, and the operability can be enhanced. According to the above structure, since the operation input direction for the first rotary body 51 and second rotary body 52 is a direction along the longitudinal axis 36, the disposition of the first rotary body 51 and second rotary body 52 can be made identical to the disposition in a conventional introduction device such as an ordinary endoscope. Thus, general therapy techniques, which have been established thus far, can be used as such for the above-described introduction device (endoscope 12), and the convenience for doctors can be improved.

The second shaft portion 63 is provided at substantially the same position as the first shaft portion 61 in the longitudinal axis 36 direction. According to this structure, the access of the thumb to the second rotary body 52 becomes good, and the work efficiency can be improved.

The operation section 32 includes the third surface 48 which is opposed to the first surface 46, and the second rotary body 52 is provided at a position between the first surface 46 and the third surface 48. According to this structure, the second rotary body 52 can be disposed within the range of the so-called thickness dimension of the operation section 32, and the access of the thumb to the second rotary body 52 can be made good.

The second surface 47 includes the recess portion 47A, and a part of the second rotary body 52 is located in the recess portion 47A. According to this structure, the height of projection of the second rotary body 52 from the second surface 47 can be decreased, and it is possible to prevent an unintentional operation of the second rotary body 52.

### Second Embodiment

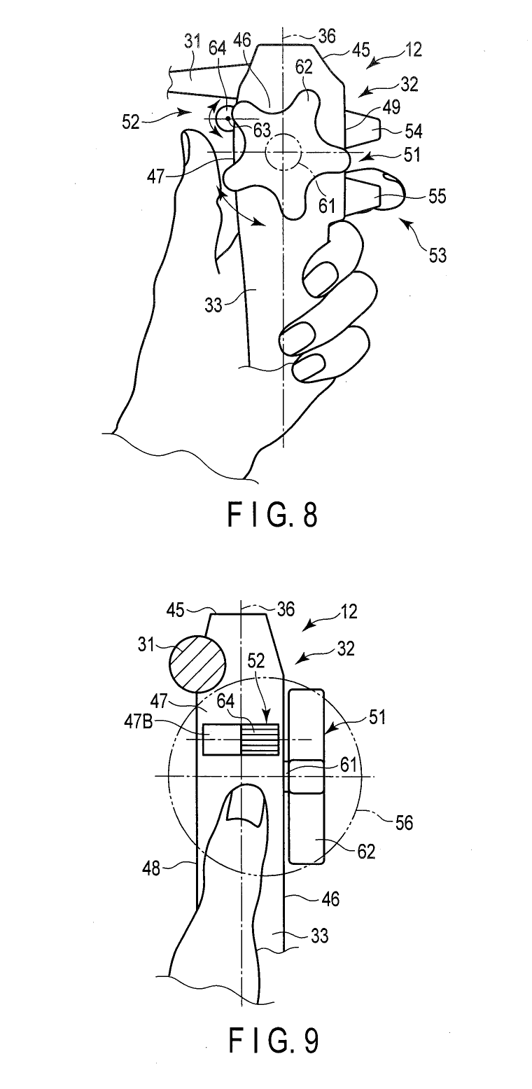

Referring to FIG. 8 and FIG. 9, an endoscope device of a second embodiment is described. The endoscope device 11 of the second embodiment differs from that of the first embodiment with respect to the position of the second shaft portion 63 of the second rotary body 52, but the other parts are common to the first embodiment. Thus, different parts from the first embodiment will mainly be described, and illustrations or descriptions of parts common to the first embodiment are omitted.

As illustrated in FIG. 8 and FIG. 9, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

The second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second rotary body 52 is provided on the second surface 47 side. The second shaft portion 63 is provided more on the proximal end side of the operation section 32 than the first shaft portion 61 in the longitudinal axis 36 direction. The second shaft portion 63 extends in a direction along the direction of extension of the first shaft portion 61 and, to be more specific, extends substantially parallel to the first shaft portion 61.

The operation of the endoscope device 11 of the present embodiment is described. When the doctor bends the bending section 38 in one of the U direction and D direction (first direction), the doctor performs the same operation as in the first embodiment.

When the doctor wishes to bend the bending section 38 in one of the R direction and L direction, the doctor rotates, as illustrated in FIG. 8, etc., the second dial portion 64 clockwise or counterclockwise by the inside of the thumb. At this time, in the present embodiment, since the second rotary body 52 (second shaft portion 63) is located more on the proximal end side of the operation section 32 than the first shaft portion 61 in the longitudinal axis 36 direction, the second rotary body 52 is prevented from being erroneously operated when not intended.

The control device 13 operates the actuator unit 21 in accordance with the rotation amount of the second rotary body 52. Thereby, the bending section 38 bends in either the R direction or L direction. In addition, if the doctor releases the thumb from the second rotary body 52, the second rotary body 52 is returned to the neutral position by the return-to-neutral mechanism 65. In accordance with this, the actuator unit 21 operates, and the bending section 38 also returns to the neutral position from the bent state in the R direction or L direction.

According to the second embodiment, the second shaft portion 63 is provided more on the proximal end side of the operation section 32 than the first shaft portion 61 in the longitudinal axis 36 direction. According to this structure, when the doctor operates the first rotary body 51, the possibility that the doctor erroneously touches the second rotary body 52 can be reduced, and an erroneous operation on the second rotary body 52 can be prevented. In addition, even if the second rotary body 52 is disposed in this manner, the second rotary body 52 is located within the operation range 56 of the thumb and is disposed at a position closer to the thumb than the first shaft portion 61. Thus, the access of the thumb to the second rotary body 52 is good, and the operability of the introduction device can be kept good.

In the meantime, in the endoscope device 11 of the present embodiment, there is no mechanism, such as a motor, for bending the bending section 38 by electric driving in the U direction and D direction. However, like the bending in the R direction and L direction, the bending section 38 may be bent in the U direction and D direction by electric driving by providing the actuator unit 21 such as a motor. In addition, in the present embodiment, although the description is given of the example in which the bending section 38 is bent by electric driving with respect to the R direction and L direction, it is possible to adopt such a configuration that the bending section 38 is manually bent in the R direction and L direction, like the bending mechanism for bending the bending section 38 in the U direction and D direction.

### Third Embodiment

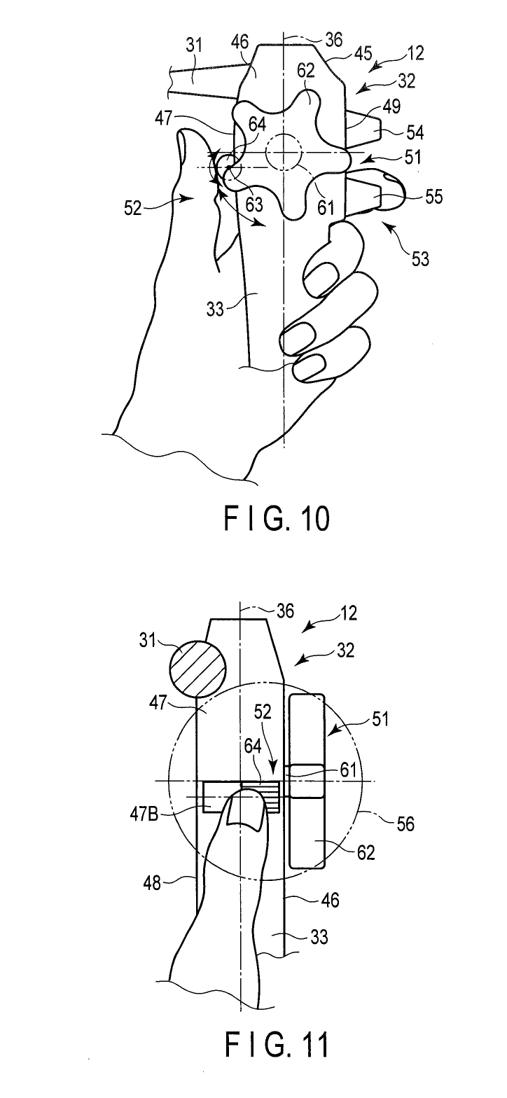

Referring to FIG. 10 and FIG. 11, an endoscope device of a third embodiment is described. The endoscope device 11 of the third embodiment differs from that of the first embodiment with respect to the position of the second rotary body 52, but the other parts are common to the first embodiment. Thus, different parts from the first embodiment will mainly be described, and illustrations or descriptions of parts common to the first embodiment are omitted.

As illustrated in FIG. 10 and FIG. 11, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

The second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second rotary body 52 is provided on the second surface 47 side. The second shaft portion 63 is provided more on the distal end side of the operation section 32 than the first shaft portion 61 in the longitudinal axis 36 direction. The second shaft portion 63 extends in a direction along the direction of extension of the first shaft portion 61 and, to be more specific, extends substantially parallel to the first shaft portion 61.

The operation of the endoscope device 11 of the present embodiment is described. When the doctor bends the bending section 38 in one of the U direction and D direction (first direction), the doctor performs the same operation as in the first embodiment.

When the doctor wishes to bend the bending section 38 in one of the R direction and L direction, the doctor rotates, as illustrated in FIG. 10, etc., the second dial portion 64 clockwise or counterclockwise by the inside of the thumb. At this time, in the present embodiment, since the second rotary body 52 (second shaft portion 63) is located more on the distal end side of the operation section 32 than the first shaft portion 61 in the longitudinal axis 36 direction, even a doctor with a small hand can naturally operate the second rotary body 52.

The control device 13 operates the actuator unit 21 in accordance with the rotation amount of the second rotary body 52. Thereby, the bending section 38 bends in either the R direction or L direction. In addition, if the doctor releases the thumb from the second rotary body 52, the second rotary body 52 is returned to the neutral position by the return-to-neutral mechanism 65. In accordance with this, the actuator unit 21 operates, and the bending section 38 also returns to the neutral position from the bent state in the R direction or L direction.

According to the third embodiment, the second shaft portion 63 is provided more on the distal end side of the operation section 32 than the first shaft portion 61 in the longitudinal axis 36 direction. According to this structure, even a doctor with a small hand (in particular, female doctor) can easily operate the second rotary body 52, and the operability of the introduction device can be improved.

In the meantime, in the endoscope device 11 of the present embodiment, there is no mechanism, such as a motor, for bending the bending section 38 by electric driving in the U direction and D direction. However, like the bending in the R direction and L direction, the bending section 38 may be bent by electric driving in the U direction and D direction by providing the actuator unit 21 such as a motor. In addition, in the present embodiment, although the description is given of the example in which the bending section 38 is bent by electric driving with respect to the R direction and L direction, it is possible to adopt such a configuration that the bending section 38 is manually bent in the R direction and L direction, like the bending mechanism for bending the bending section 38 in the U direction and D direction.

### Fourth Embodiment

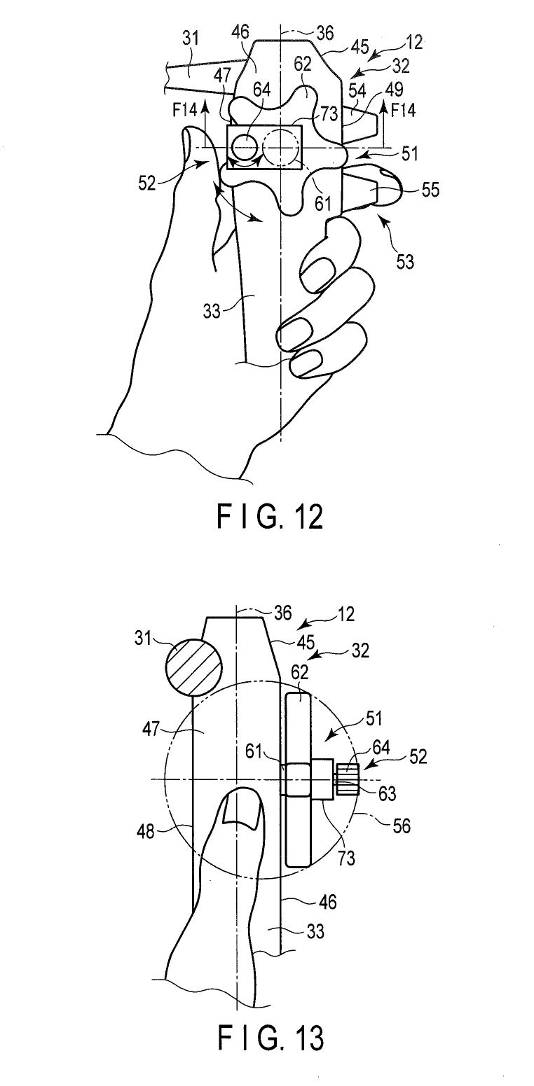

Referring to FIG. 12 to FIG. 14, an endoscope device of a fourth embodiment is described. The endoscope device 11 of the fourth embodiment differs from that of the first embodiment with respect to the position of disposition of the second rotary body 52, but the other parts are common to the first embodiment. Thus, different parts from the first embodiment will mainly be described, and illustrations or descriptions of parts common to the first embodiment are omitted.

As illustrated in FIG. 12 and FIG. 13, the second rotary body 52 is provided near the first rotary body 51 on the second surface 47 side, and is provided within the operation range 56 of the thumb of the left hand that holds the operation section 32. Specifically, the second rotary body 52 is provided at a position farther from the thumb than the first rotary body 51. In other words, although the second rotary body 52 is located outside the range of the thickness dimension of the case 45, the second rotary body 52 is provided on the second surface 47 side. In the present embodiment, the first rotary body 51 is provided at a position between the second rotary body 52 and the first surface 46 (case 45).

As illustrated in FIG. 12 to FIG. 14, the second rotary body 52 includes a second shaft portion 63 which is located in the inside of the first shaft portion 61 of the first rotary body 51 and is rotatable relative to the case 45; a cylindrical portion 72 which is interposed between the second shaft portion 63 and first shaft portion 61 and is fixed to a frame 71 which is built in the operation section 32; a support portion 73 which is fixed to a distal end of the cylindrical portion 72; a second dial portion 64 which is provided on the support portion 73 and is rotatable relative to the support portion 73; a pair of gear portions 74 which are built in the support portion 73 and is interposed between the second dial portion 64 and second shaft portion 63; and a return-to-neutral mechanism 65 which applies a return-to-neutral force to the second shaft portion 63.

The second shaft portion 63 is provided at substantially the same position as the first shaft portion 61 in the longitudinal axis 36 direction. The second shaft portion 63 extends in a direction along the direction of extension of the first shaft portion 61 and, to be more specific, extends substantially parallel to the first shaft portion 61.

The operation of the endoscope device 11 of the present embodiment is described. When the doctor bends the bending section 38 in one of the U direction and D direction (first direction), the doctor performs the same operation as in the first embodiment.

When the doctor wishes to bend the bending section 38 in one of the R direction and L direction, the doctor rotates, as illustrated in FIG. 12, etc., the second dial portion 64 clockwise or counterclockwise by the inside of the thumb. At this time, in the present embodiment, since the second rotary body 52 is located on the second surface 47 side within the operation range 56 of the thumb of the left hand, the operability on the second rotary body 52 becomes good.

The control device 13 operates the actuator unit 21 in accordance with the rotation amount of the second rotary body 52. Thereby, the bending section 38 bends in either the R direction or L direction. In addition, if the doctor releases the thumb from the second rotary body 52, the second rotary body 52 is returned to the neutral position by the return-to-neutral mechanism 65. In accordance with this, the actuator unit 21 operates, and the bending section 38 also returns to the neutral position from the bent state in the R direction or L direction.

According to the fourth embodiment, the first rotary body 51 is provided at a position between the second rotary body 52 and the first surface 46. According to this structure, although the second rotary body 52 is located outside the range of the thickness dimension of the case 45, the second rotary body 52 is provided within the operation range 56 of the thumb of the holding hand on the second surface 47 side. Thus, the operability of the second rotary body 52 can be kept good, and an erroneous operation of the second rotary body 52 can be prevented. The present embodiment is particularly useful when there is no space for providing the second rotary body 52 within the thickness dimension of the case 45, and when the second rotary body 52 is to be provided on the second surface 47 side.

In the meantime, in the endoscope device 11 of the present embodiment, there is no mechanism, such as a motor, for bending the bending section 38 by electric driving in the U direction and D direction. However, like the bending in the R direction and L direction, the bending section 38 may be bent by electric driving in the U direction and D direction by providing the actuator unit 21 such as a motor. In addition, in the present embodiment, although the description is given of the example in which the bending section 38 is bent by electric driving with respect to the R direction and L direction, it is possible to adopt such a configuration that the bending section 38 is manually bent in the R direction and L direction, like the bending mechanism for bending the bending section 38 in the U direction and D direction.

### Fifth Embodiment

Referring to FIG. 15 to FIG. 17, an endoscope device of a fifth embodiment is described. The endoscope device 11 of the fifth embodiment differs from that of the first embodiment with respect to the position of the rotation detection sensor 26, but the other parts are common to the first embodiment. Thus, different parts from the first embodiment will mainly be described, and illustrations or descriptions of parts common to the first embodiment are omitted.

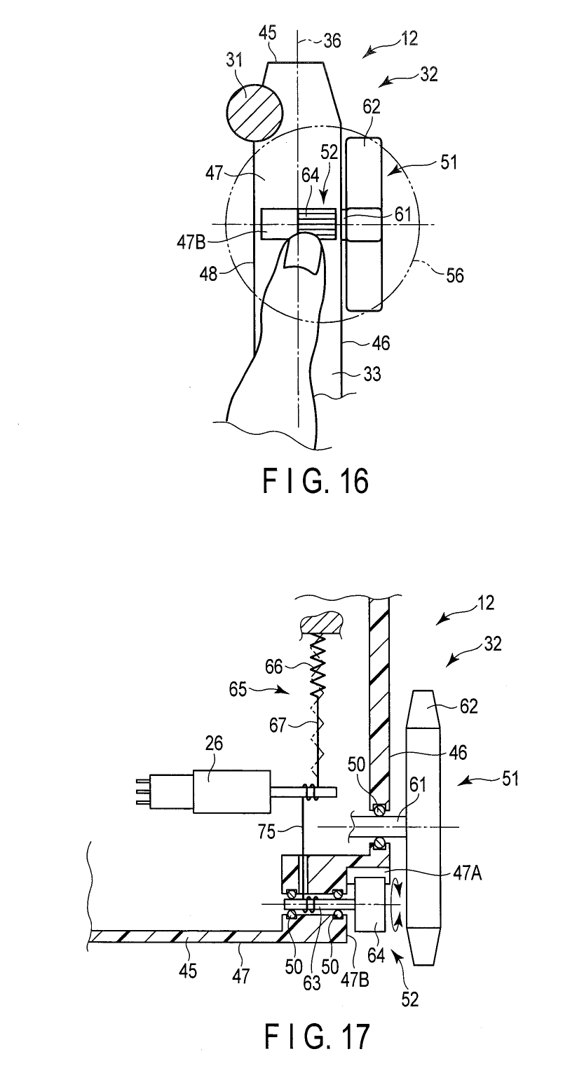

As illustrated in FIG. 15 and FIG. 16, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

Unlike the first embodiment, the rotation detection sensor 26 is provided at a position displaced from the second rotary body 52 in the inside of the operation section 32. As illustrated in FIG. 17, a wire 75 for rotation detection is interposed between the second rotary body 52 and the rotation detection sensor 26. The rotation amount of the second rotary body 52 can be detected by the rotation detection sensor 26 via the wire 75 for rotation detection. A belt, a gear, etc. may be substituted for the wire 75 for rotation detection.

In addition, a return-to-neutral mechanism 65 (tension coil spring 66) is connected to the rotation detection sensor 26 via a tension wire 67. When the doctor has released the finger from the second rotary body 52, the return-to-neutral mechanism 65 can return the rotation detection sensor 26 and the second rotary body 52, which is connected to the rotation detection sensor 26, to the neutral position.

The operation of the endoscope device 11 of the present embodiment is described. When the doctor bends the bending section 38 in one of the U direction and D direction (first direction), the doctor performs the same operation as in the first embodiment. When the doctor wishes to bend the bending section 38 in one of the R direction and L direction, the doctor rotates, as illustrated in FIG. 15, etc., the second dial portion 64 clockwise or counterclockwise by the inside of the thumb.

The control device 13 operates the actuator unit 21 in accordance with the rotation amount of the second rotary body 52, which was detected by the rotation detection sensor 26. Thereby, the bending section 38 bends in either the R direction or L direction. In addition, if the doctor releases the thumb from the second rotary body 52, the rotation detection sensor 26 and the second rotary body 52 are returned to the neutral position by the return-to-neutral mechanism 65. In accordance with this, the actuator unit 21 operates, and the bending section 38 also returns to the neutral position from the bent state in the R direction or L direction.

According to the fifth embodiment, the degree of freedom of layout of the rotation detection sensor 26 within the operation section 32 can be improved. Thereby, miniaturization of the operation section 32 can be advanced, and it is possible to realize the introduction device including the operation section 32 which is easy to hold even by a doctor with a small hand, and which has good operability.

In the meantime, in the endoscope device 11 of the present embodiment, there is no mechanism, such as a motor, for bending the bending section 38 by electric driving in the U direction and D direction. However, like the bending in the R direction and L direction, the bending section 38 may be bent by electric driving in the U direction and D direction by providing the actuator unit 21 such as a motor. In addition, in the present embodiment, although the description is given of the example in which the bending section 38 is bent by electric driving with respect to the R direction and L direction, it is possible to adopt such a configuration that the bending section 38 is manually bent in the R direction and L direction, like the bending mechanism for bending the bending section 38 in the U direction and D direction.

### Sixth Embodiment

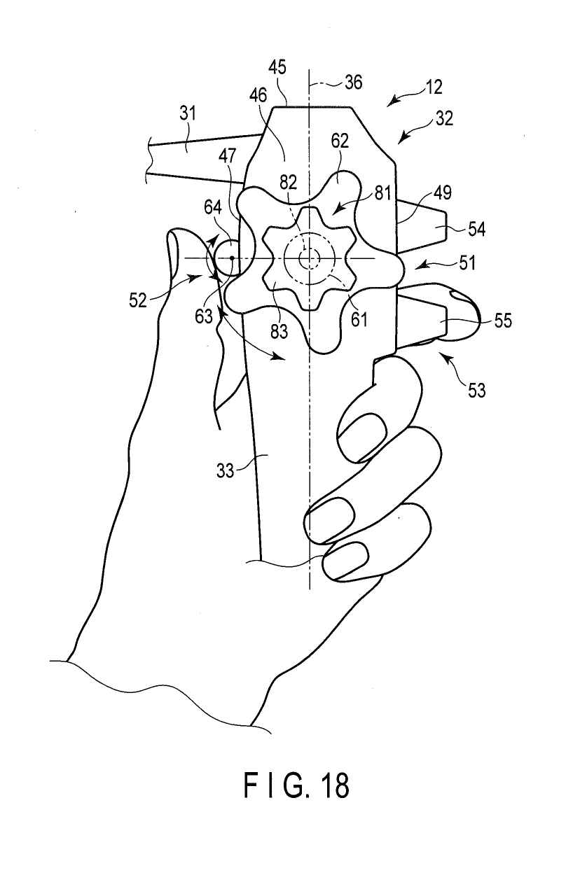

Referring to FIG. 18 to FIG. 20, an endoscope device of a sixth embodiment is described. The endoscope device 11 of the sixth embodiment differs from that of the first embodiment with respect to the provision of a third rotary body 81 and the operations of the bending section 38, which correspond to the first rotary body 51 and second rotary body 52, but the other parts are common to the first embodiment. Thus, different parts from the first embodiment will mainly be described, and illustrations or descriptions of parts common to the first embodiment are omitted.

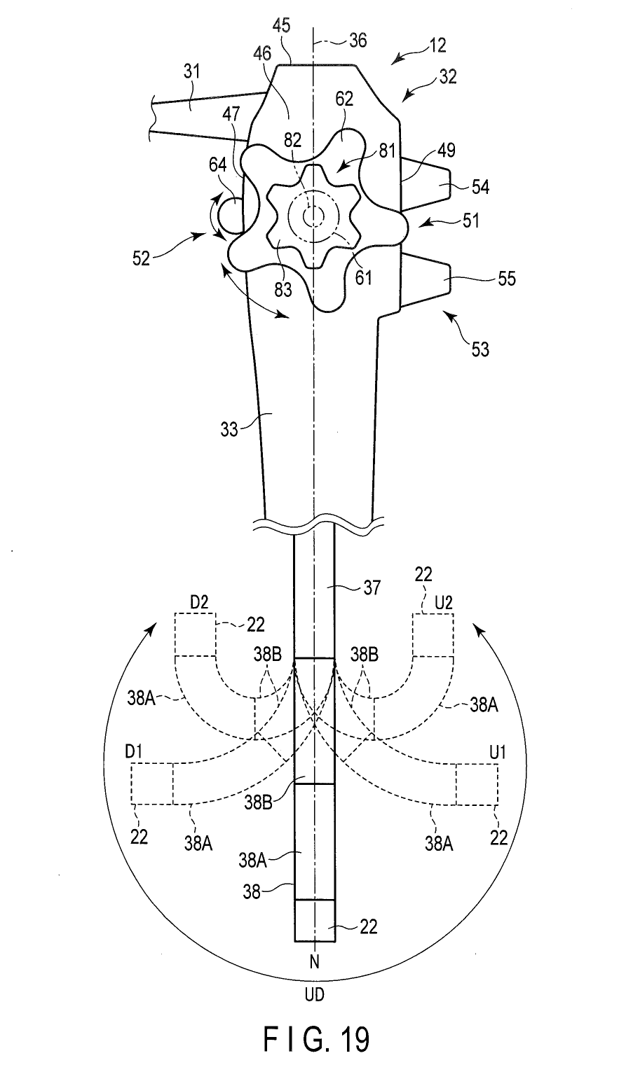

As illustrated in FIG. 19, in the present embodiment, the bending section 38 includes a first part 38A which is disposed on a distal end side of the longitudinal axis 36 direction, and a second part 38B which is disposed on a proximal end side of the longitudinal axis 36 direction.

The first rotary body 51 is a so-called first UD knob which is operated at a time of bending the first part 38A of the bending section 38 in the U direction and D direction. If the doctor rotates the first rotary body 51 about the first shaft portion 61 by a finger or the like, at least the first part 38A of the bending section 38 is bent in accordance with the rotation amount in the U direction or D direction (these directions are comprehensively referred to as “first direction”), as illustrated in FIG. 19.

As illustrated in FIG. 18, the first rotary body 51 includes a first shaft portion 61 which projects from the first surface 46 of the case 45 of the operation section 32 and is provided rotatable relative to the case 45; a first dial portion 62 which is fixed to a distal end portion of the first shaft portion 61 and is rotatable about the first shaft portion 61; and a first sprocket 35 (see FIG. 14) which is provided within the case 45 and is fixed to a proximal end portion of the first shaft portion 61. The first wires 41 for bending the bending section 38 in the U direction and D direction are wound around the first sprocket 35. In the present embodiment, the doctor bends the first part 38A of the bending section 38 in the U direction and D direction by the force of the finger.

The second rotary body 52 is a so-called second UD dial which is operated at a time of bending the second part 38B of the bending section 38 in the U direction and D direction. If the doctor rotates the second rotary body 52 about the second shaft portion 63, the actuator unit 21 is driven, and the second part 38B of the bending section 38 is bent by the driving force of the actuator unit 21 via a pair of wires which are disposed substantially parallel to the first wires 41. In accordance with the rotation amount of the second rotary body 52, the second part 38B of the bending section 38 is bent by electric driving in the U direction and D direction (these directions are comprehensively referred to as “second direction”) illustrated in FIG. 19.

In the present embodiment, the second direction, which corresponds to the operation of the second rotary body 52, agrees with the first direction which corresponds to the operation of the first rotary body 51. In the meantime, in the state in which the first part 38A of the bending section 38 is bent by operating the first rotary body 51, if the second part 38B of the bending section 38 is further bent by operating the second rotary body 52, the bending section 38 is set in a state in which the bending section 38 is bent to a maximum degree in the U direction or D direction, as illustrated in FIG. 19.

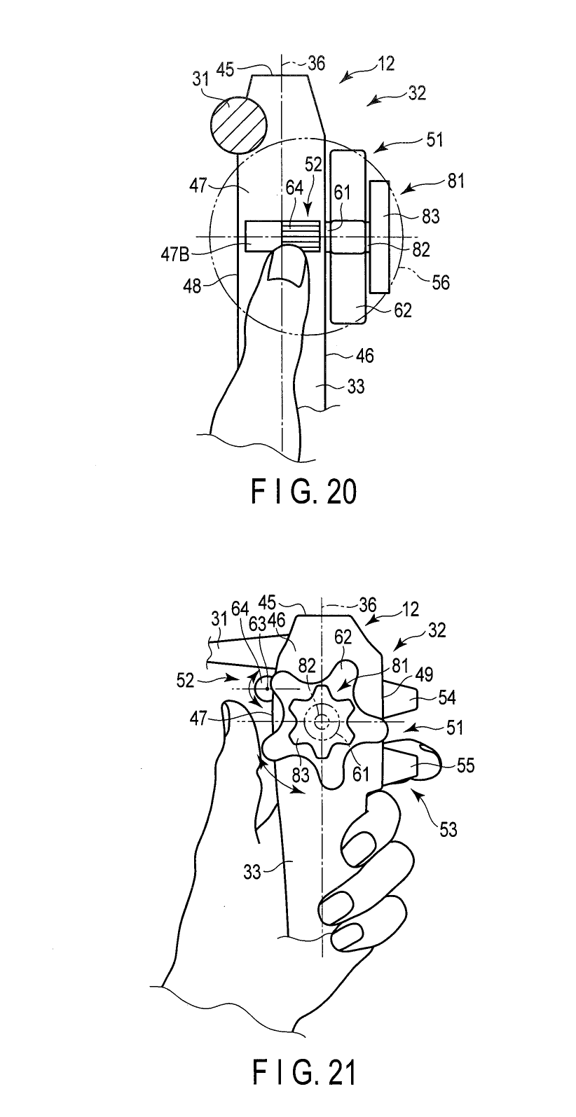

As illustrated in FIG. 18 and FIG. 20, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is provided within the range of the thickness dimension of the case 45 in the direction of extension of the first shaft portion 61.

The second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second rotary body 52 is provided on the second surface 47 side. The second shaft portion 63 is provided at substantially the same position as the first shaft portion 61 in the longitudinal axis 36 direction. The second shaft portion 63 extends in a direction along the direction of extension of the first shaft portion 61 and, to be more specific, extends substantially parallel to the first shaft portion 61.

The third rotary body 81 is a so-called RL knob which is operated at a time of bending the first part 38A of the bending section 38 in the R direction and L direction. If the doctor rotates the third rotary body 81 about a third shaft portion 82 by a finger or the like, at least the first part 38A of the bending section 38 is bent in accordance with the rotation amount in the R direction or L direction (these directions are comprehensively referred to as “third direction”) illustrated in FIG. 2. In the present embodiment, the third direction crosses (interests at right angles with) the first direction (second direction).

The third rotary body 81 includes a third shaft portion 82 which projects from the first surface 46 of the case 45 of the operation section 32 and is provided rotatable relative to the case 45; a third dial portion 83 which is fixed to a distal end portion of the third shaft portion 82 and is rotatable about the third shaft portion 82; and a third sprocket 35 (not shown) which is provided within the case 45 and is fixed to a proximal end portion of the third shaft portion 82. Second wires 42 for bending the bending section 38 in the R direction and L direction are wound around the third sprocket. In the present embodiment, the doctor bends the bending section 38 in the R direction and L direction by the force of the finger.

The operation of the endoscope device 11 of the present embodiment is described. When the doctor wishes to bend the bending section 38 in one of the U direction and D direction (first direction) illustrated in FIG. 2, the doctor rotates the first dial portion 62 clockwise or counterclockwise by the inside of the thumb of the left hand. Thereby, the first sprocket 35, which is fixed to the first shaft portion 61 in the inside of the operation section 32, rotates, and one of the paired first wires 41, which are wound around the first sprocket 35, is pulled toward the proximal end side of the operation section 32. Thus, at least the first part 38A of the bending section 38 bends in either the U direction or D direction. Specifically, as illustrated in FIG. 19, if the first rotary body 51 is rotated clockwise, the first part 38A of the bending section 38 bends in the D (downward) direction, and the bending section 38 moves from a neutral position N to a D-side normal bent position D1. If the first rotary body 51 is rotated counterclockwise, the first part 38A of the bending section 38 bends in the U (upward) direction, and the bending section 38 moves from the neutral position N to a U-side normal bent position U1.

If the doctor wishes to further bend the bending section 38 to a maximum bent state in one of the U direction and D direction (first direction), the doctor rotates the second dial portion 64 clockwise or counterclockwise by the inside of the thumb of the left hand. The control device 13 operates the actuator unit 21 in accordance with the rotation amount of the second rotary body 52. The driving force of the actuator unit 21 is transmitted, via the flexible shaft and the gear and sprocket provided within the operation unit 32, to the paired wires which are wound around this sprocket. By one of the paired wires being pulled, the second part 38B of the bending section 38 bends in either the U direction or D direction. Thus, the bending section 38 is set in the maximum bent state (D-side maximum bent position D2, U-side maximum bent position U2) in combination with the bending at the first part 38A. In addition, if the doctor releases the thumb from the second rotary body 52, the second rotary body 52 is returned to the neutral position by the return-to-neutral mechanism 65. In accordance with this, the actuator unit 21 operates, and the second part 38B of the bending section 38 also returns to the neutral position from the bent state in the U direction or D direction.

On the other hand, when the doctor wishes to bend the bending section 38 in one of the R direction and L direction, the doctor rotates the third dial portion 83 clockwise or counterclockwise by the inside of the thumb. Thereby, the third sprocket, which is fixed to the third shaft portion 82 in the inside of the operation section 32, rotates, and one of the paired second wires 42, which are wound around this third sprocket, is pulled to the proximal end side of the operation section 32. Thus, at least the first part 38A of the bending section 38 bends in one of the U direction and D direction.

According to the present embodiment, the bending section 38 includes the first part 38A on the distal end side and the second part 38B on the proximal end side, and the second direction agrees with the first direction. The first rotary body 51 is operated at the time of bending at least the first part 38A of the bending section 38 in the first direction, and the second rotary body 52 is operated at the time of bending at least the second part 38B of the bending section 38 in the second direction. According to this structure, even when the first rotary body 51 and second rotary body 52 bend the bending section 38 in the same direction, the operability of the second rotary body 52 can be made good.

In the meantime, in the endoscope device 11 of the present embodiment, no motor or the like exists in the mechanism for bending the bending section 38 in the U direction and D direction in association with the first rotary body 51 or in the mechanism for bending the bending section 38 in the R direction and L direction in association with the third rotary body 81. However, in these mechanisms, the actuator unit 21, such as a motor, may be provided, like the mechanism for bending the bending section 38 in the U direction and D direction in association with the second rotary body 52. Besides, a mechanism for manually bending the bending section 38 in the U direction or D direction may be used for the mechanism for bending the bending section 38 in the U direction and D direction in association with the second rotary body 52.

### Seventh Embodiment

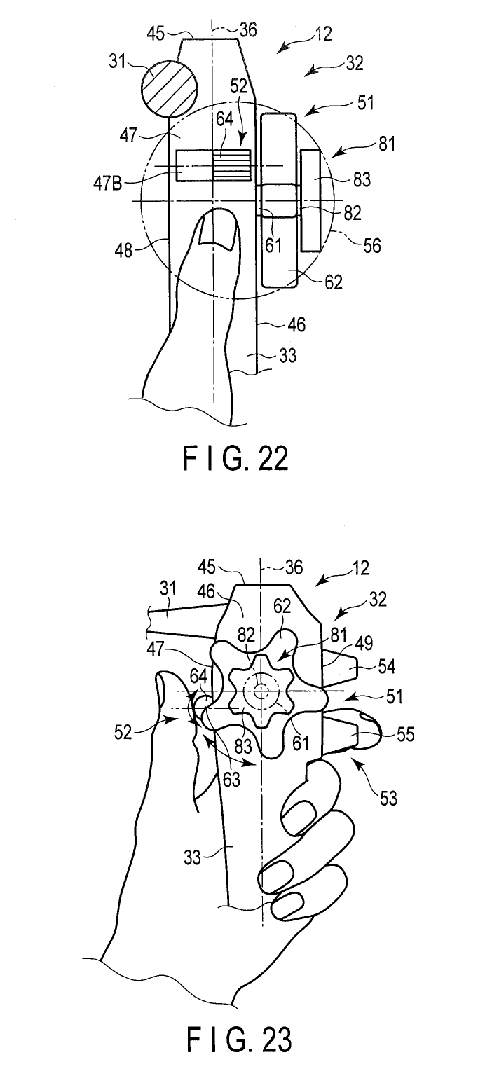

Referring to FIG. 21 to FIG. 22, an endoscope device of a seventh embodiment is described. The endoscope device 11 of the seventh embodiment differs from that of the sixth embodiment with respect to the position of the second rotary body 52, but the other parts are common to the sixth embodiment. Thus, different parts from the sixth embodiment will mainly be described, and illustrations or descriptions of parts common to the sixth embodiment are omitted.

As illustrated in FIG. 21 and FIG. 22, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

The second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second rotary body 52 is provided on the second surface 47 side. The second shaft portion 63 is provided more on the proximal end side of the operation section 32 than the first shaft portion 61 in the longitudinal axis 36 direction. The second shaft portion 63 extends in a direction along the direction of extension of the first shaft portion 61 and, to be more specific, extends substantially parallel to the first shaft portion 61.

The operation of the endoscope device 11 of the present embodiment is described. When the doctor wishes to bend the bending section 38 in one of the U direction and D direction (first direction) illustrated in FIG. 2, the doctor rotates the first dial portion 62 clockwise or counterclockwise by the inside of the thumb of the left hand. Thereby, the first sprocket 35, which is fixed to the first shaft portion 61 in the inside of the operation section 32, rotates, and one of the paired first wires 41, which are wound around the first sprocket 35, is pulled toward the proximal end side of the operation section. Thus, at least the first part 38A of the bending section 38 bends in either the U direction or D direction.

If the doctor wishes to further bend the bending section 38 to a maximum bent state in one of the U direction and D direction (first direction), the doctor rotates the second dial portion 64 clockwise or counterclockwise by the inside of the thumb of the left hand. The control device 13 operates the actuator unit 21 in accordance with the rotation amount of the second rotary body 52. Thereby, the second part 38B of the bending section 38 bends in the U direction or D direction, and the bending section 38 is set in the maximum bent state in combination with the bending at the first part 38A. At this time, in the present embodiment, since the second rotary body is provided more on the proximal end side of the operation section 32 than the first shaft portion in the longitudinal axis direction, the second rotary body 52 is prevented from being erroneously operated when not intended.

In addition, if the doctor releases the thumb from the second rotary body 52, the second rotary body 52 is returned to the neutral position by the return-to-neutral mechanism 65. In accordance with this, the actuator unit 21 operates, and the second part 38B of the bending section 38 also returns to the neutral position from the bent state in the U direction or D direction.

On the other hand, when the doctor wishes to bend the bending section 38 in one of the R direction and L direction, the doctor performs the same operation as in the sixth embodiment.

According to the seventh embodiment, in the introduction device, the second shaft portion 63 is provided more on the proximal end side of the operation section 32 than the first shaft portion 61 in the longitudinal axis 36 direction. According to this structure, when the doctor operates the first rotary body 51, the possibility that the doctor erroneously touches the second rotary body 52 can be reduced, and an erroneous operation on the second rotary body 52 can be prevented. In addition, even if the second rotary body 52 is disposed in this manner, the second rotary body 52 is located within the operation range 56 of the thumb and is disposed at a position closer to the thumb than the first shaft portion 61. Thus, the access of the thumb to the second rotary body 52 is good, and the operability of the introduction device can be kept good.

In the meantime, in the endoscope device 11 of the present embodiment, no motor or the like exists in the mechanism for bending the bending section 38 in the U direction and D direction in association with the first rotary body 51 or in the mechanism for bending the bending section 38 in the R direction and L direction in association with the third rotary body 81. However, in these mechanisms, the actuator unit 21, such as a motor, may be provided, like the mechanism for bending the bending section 38 in the U direction and D direction in association with the second rotary body 52. Besides, a mechanism for manually bending the bending section 38 in the U direction or D direction may be used for the mechanism for bending the bending section 38 in the U direction and D direction in association with the second rotary body 52.

### Eighth Embodiment

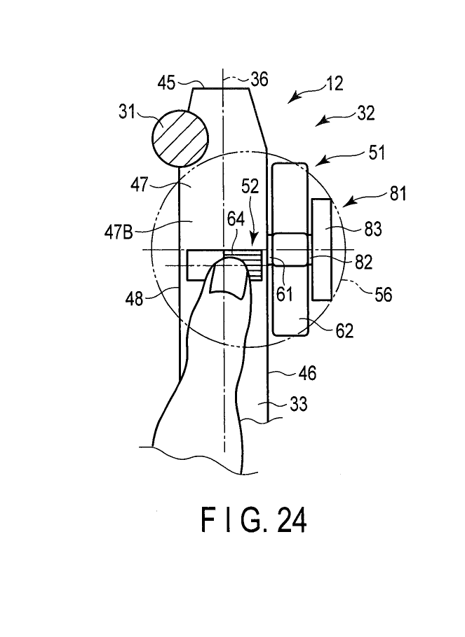

Referring to FIG. 23 to FIG. 24, an endoscope device of an eighth embodiment is described. The endoscope device of the eighth embodiment differs from that of the sixth embodiment with respect to the position of the second rotary body 52, but the other parts are common to the sixth embodiment. Thus, different parts from the sixth embodiment will mainly be described, and illustrations or descriptions of parts common to the sixth embodiment are omitted.

As illustrated in FIG. 23 and FIG. 24, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

The second rotary body 52 includes a second shaft portion 63 which projects from a projection portion 47B provided on the second surface 47 of the operation section 32 and is rotatable relative to the case 45; a second dial portion 64 fixed to a distal end portion of the second shaft portion 63; and a return-to-neutral mechanism 65 which applies a return-to-neutral force to the second shaft portion 63. The second shaft portion 63 is provided more on the distal end side of the operation section 32 than the first shaft portion 61 in the longitudinal axis 36 direction. The second shaft portion 63 extends in a direction along the direction of extension of the first shaft portion 61 and, to be more specific, extends substantially parallel to the first shaft portion 61.

The operation of the endoscope device 11 of the present embodiment is described. When the doctor wishes to bend the bending section 38 in one of the U direction and D direction (first direction) illustrated in FIG. 2, the doctor rotates the first dial portion 62 clockwise or counterclockwise by the inside of the thumb of the left hand. Thereby, the first sprocket 35, which is fixed to the first shaft portion 61 in the inside of the operation section 32, rotates, and one of the paired first wires 41, which are wound around the first sprocket 35, is pulled toward the proximal end side of the operation section 32. Thus, at least the first part 38A of the bending section 38 bends in either the U direction or D direction.

If the doctor wishes to further bend the bending section 38 to a maximum bent state in one of the U direction and D direction (first direction), the doctor rotates the second dial portion 64 clockwise or counterclockwise by the inside of the thumb of the left hand. The control device 13 operates the actuator unit 21 in accordance with the rotation amount of the second rotary body 52. Thereby, the second part 38B of the bending section 38 bends in the U direction or D direction, and the bending section 38 is set in the maximum bent state in combination with the bending at the first part 38A. At this time, in the present embodiment, since the second rotary body 52 is provided more on the distal end side of the operation section 32 than the first shaft portion 61 in the longitudinal axis 36 direction, even a doctor with a small hand can naturally operate the second rotary body 52.

In addition, if the doctor releases the thumb from the second rotary body 52, the second rotary body 52 is returned to the neutral position by the return-to-neutral mechanism 65. In accordance with this, the actuator unit 21 operates, and the second part 38B of the bending section 38 also returns to the neutral position from the bent state in the U direction or D direction.

On the other hand, when the doctor wishes to bend the bending section 38 in one of the R direction and L direction, the doctor performs the same operation as in the sixth embodiment.

According to the eighth embodiment, in the introduction device, the second shaft portion 63 is provided more on the distal end side of the operation section 32 than the first shaft portion 61 in the longitudinal axis 36 direction. According to this structure, even a doctor with a small hand (in particular, female doctor) can easily operate the second rotary body 52, and the operability of the introduction device can be improved.

In the meantime, in the endoscope device 11 of the present embodiment, no motor or the like exists in the mechanism for bending the bending section 38 in the U direction and D direction in association with the first rotary body 51 or in the mechanism for bending the bending section 38 in the R direction and L direction in association with the third rotary body 81. However, in these mechanisms, the actuator unit 21, such as a motor, may be provided, like the mechanism for bending the bending section 38 in the U direction and D direction in association with the second rotary body 52. Besides, a mechanism for manually bending the bending section 38 in the U direction or D direction may be used for the mechanism for bending the bending section 38 in the U direction and D direction in association with the second rotary body 52.

### Ninth Embodiment

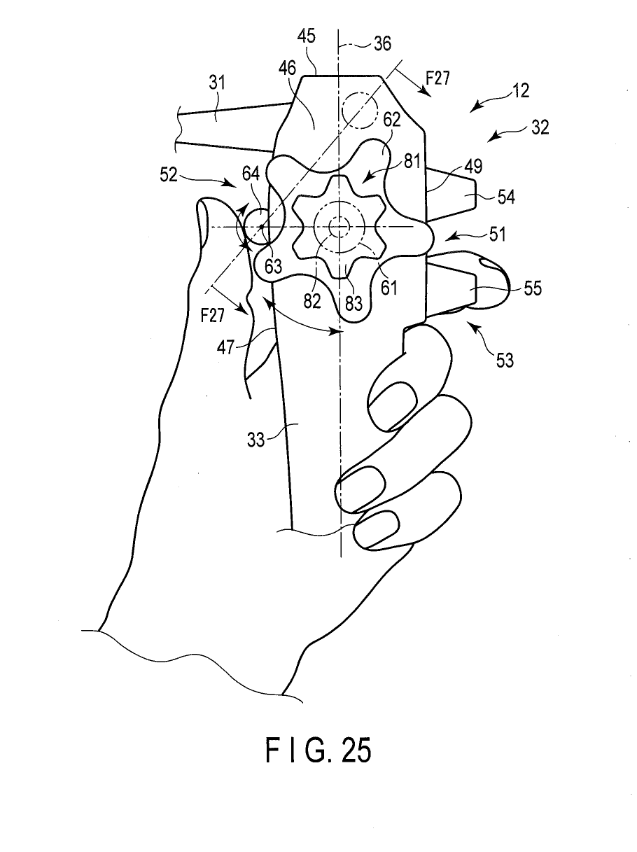

Referring to FIG. 25 to FIG. 27, an endoscope device of a ninth embodiment is described. The endoscope device 11 of the ninth embodiment differs from that of the sixth embodiment with respect to the position of the rotation detection sensor 26, but the other parts are common to the sixth embodiment. Thus, different parts from the sixth embodiment will mainly be described, and illustrations or descriptions of parts common to the sixth embodiment are omitted.

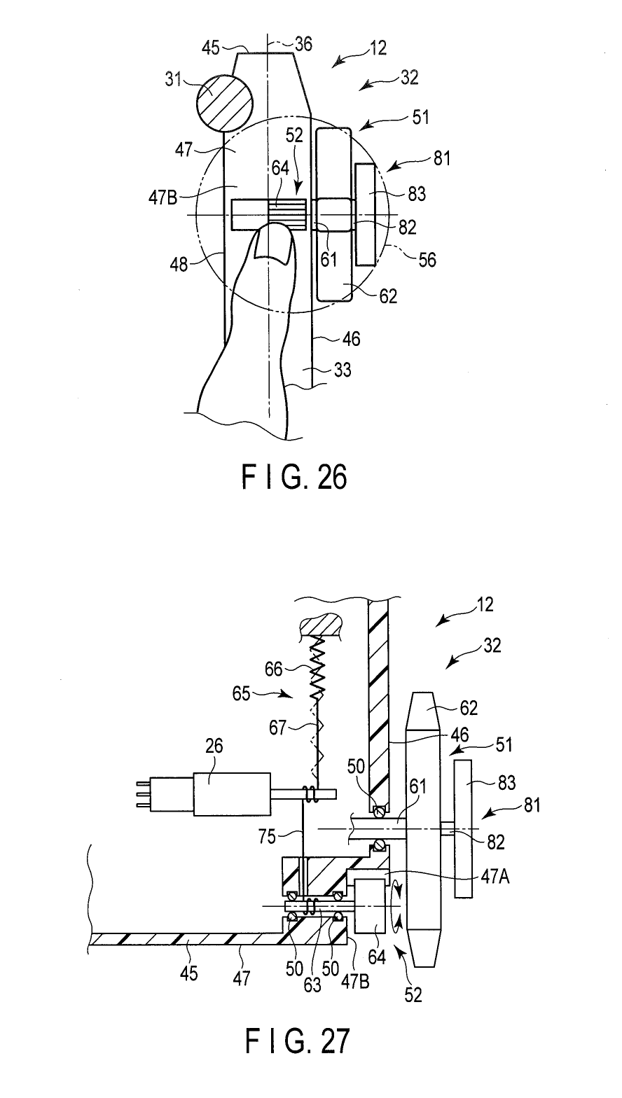

As illustrated in FIG. 25 and FIG. 26, the second rotary body 52 is provided near the first rotary body 51 on the second surface 47 side, and is provided within the operation range 56 of the thumb of the left hand that holds the operation section 32. In this embodiment, the second rotary body 52 is provided at a position between the first surface 46 and third surface 48. In other words, the second rotary body 52 is located within the range of the thickness dimension of the case 45 in the direction of extension of the first shaft portion 61.

Unlike the sixth embodiment, the rotation detection sensor 26 is provided at a position displaced from the second rotary body 52 in the inside of the operation section 32. A wire 75 for rotation detection is interposed between the second rotary body 52 and the rotation detection sensor 26. The rotation amount of the second rotary body 52 can be detected by the rotation detection sensor 26 via the wire 75 for rotation detection. A belt, a gear, etc. may be substituted for the wire 75 for rotation detection.

In addition, a return-to-neutral mechanism 65 is connected to the rotation detection sensor 26 via another wire. When the doctor has released the finger from the second rotary body 52, the return-to-neutral mechanism 65 can return the rotation detection sensor 26 and the second rotary body 52, which is connected to the rotation detection sensor 26, to the neutral position.

The operation of the endoscope device 11 of the present embodiment is described. When the doctor wishes to bend the bending section 38 in one of the U direction and D direction (first direction) illustrated in FIG. 2, the doctor rotates the first dial portion 62 clockwise or counterclockwise by the inside of the thumb of the left hand. Thereby, the first sprocket 35, which is fixed to the first shaft portion 61 in the inside of the operation section 32, rotates, and one of the paired first wires 41, which are wound around the first sprocket 35, is pulled toward the proximal end side of the operation section 32. Thus, at least the first part 38A of the bending section 38 bends in either the U direction or D direction.

If the doctor wishes to further bend the bending section 38 to a maximum bent state in one of the U direction and D direction (first direction), the doctor rotates the second dial portion 64 clockwise or counterclockwise by the inside of the thumb of the left hand. The control device 13 operates the actuator unit 21 in accordance with the rotation amount of the second rotary body 52. Thereby, the second part 38B of the bending section 38 bends in the U direction or D direction, and the bending section 38 is set in the maximum bent state in combination with the bending at the first part 38A. At this time, in the present embodiment, since the second rotary body 52 is provided more on the distal end side of the operation section 32 than the first shaft portion 61 in the longitudinal axis 36 direction, even a doctor with a small hand can naturally operate the second rotary body 52.

In addition, if the doctor releases the thumb from the second rotary body 52, the second rotary body 52 is returned to the neutral position by the return-to-neutral mechanism 65. In accordance with this, the actuator unit 21 operates, and the second part 38B of the bending section 38 also returns to the neutral position from the bent state in the U direction or D direction.

On the other hand, when the doctor wishes to bend the bending section. 38 in one of the R direction and L direction, the doctor performs the same operation as in the sixth embodiment.

According to the ninth embodiment, the degree of freedom of layout of the rotation detection sensor 26 within the operation section 32 can be improved. Thereby, miniaturization of the operation section 32 can be advanced, and it is possible to realize the introduction device including the operation section which is easy to hold even by a doctor with a small hand, and which has good operability.

In the meantime, in the endoscope device of the present embodiment, no motor or the like exists in the mechanism for bending the bending section 38 in the U direction and D direction in association with the first rotary body 51 or in the mechanism for bending the bending section 38 in the R direction and L direction in association with the third rotary body 81. However, in these mechanisms, the actuator unit, such as a motor, may be provided, like the mechanism for bending the bending section 38 in the U direction and D direction in association with the second rotary body 52. Besides, a mechanism for manually bending the bending section 38 in the U direction or D direction may be used for the mechanism for bending the bending section 38 in the U direction and D direction in association with the second rotary body 52.

The present invention is not limited to the above-described embodiments, and modifications may be implemented where necessary, without departing from the spirit of the invention. In addition, one endoscope device may be composed by combining the endoscope devices of the above-described embodiments.

In each of the above-described embodiments, the endoscope is used as an example of the introduction device. Other examples of the introduction device include an introduction device which does not include the illumination optical system including the light source device 14 and illumination lenses 23 of the distal rigid section 22, or the observation optical system including the image capturing device 15, monitor 18 and the objective lens 25 of the distal rigid section 22.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. An introduction device comprising:
a bending section capable of bending in a first direction and a second direction crossing the first direction;
an operation section held to allow a bending operation of the bending section, including a first surface, and a second surface which neighbors the first surface and is located on a side of a thumb of a holding hand;
a first rotary body configured to be rotatable about a first shaft portion provided on the first surface in a direction crossing a longitudinal axis of the operation section, and to be operated to bend the bending section in the first direction; and
a second rotary body configured to be rotatable about a second shaft portion extending in a direction along the first shaft portion at a position which is closer to the thumb than the first shaft portion and at a position which is closer to the distal end of the operation section than the first shaft portion in the direction of the longitudinal axis, to be provided on the second surface side within an operation range of the thumb, and to be operated to bend the bending section in the second direction.

2-8. (canceled)

9. The introduction device of claim 1, wherein the operation section includes a third surface which is opposed to the first surface, and
the second rotary body is provided at a position between the first surface and the third surface.

10. The introduction device of claim 1, wherein the second surface includes a recess portion, and
a part of the second rotary body is located in the recess portion.

11. (canceled)

12. The introduction device of claim 1, wherein the second shaft portion is substantially parallel to the first shaft portion.

13. The introduction device of claim 1, further comprising an actuator configured to generate a driving force for bending the bending section in the second direction, and rotation detection means for detecting a rotation amount of the second rotary body,
wherein, based on the rotation amount of the second rotary body, which the rotation detection means detects, the actuator is caused to generate the driving force and to bend the bending section in the second direction.

14. An introduction device comprising:
a bending section that comprises a first part on a distal end and a second part on a proximal end and that is bendable;
an operation section being held to allow a bending operation of the bending section and comprising a first surface and a second surface adjacent to the first surface, located on the side of the thumb of the holding hand;
a first rotary body being rotatable about a first shaft portion provided on the first surface in the direction crossing the longitudinal axis of the operation section, and operated to bend the first part of the bending section; and
a second rotary body being rotatable at a position which is closer to the thumb than the first shaft portion and closer to the distal end of the operation section than the first shaft portion in the direction of the longitudinal axis about a second shaft portion extending in the direction along the first shaft portion, provided within an operation range of the thumb on the second surface, and operated to bend the second part of the bending section.
